(19) United States
(12) Patent Application Publication
(10) Pub. No.: US 2005/0203561 A1
(43) Pub. Date: Sep. 15, 2005

(54) Lighted dissector and method for use

(76) Inventors: Joetta Renee Palmer, Mason, OH (US); Randall Kevin Wolf, Cincinnati, OH (US); Eric William Schneeberger, Cincinnati, OH (US); Patrick Jerome Alexander, Cincinnati, OH (US); Daniel William Divelbiss, Fredericktown, OH (US); Matthew Joseph Winkler, Cincinnati, OH (US); Adam Ray Harp, Cincinnati, OH (US); Richard Paul Nuchols, Loveland, OH (US)

Correspondence Address: FROST BROWN TODD, LLC, 2200 PNC CENTER, 201 E. FIFTH STREET, CINCINNATI, OH, 45202, US

(21) Appl. No.: 10/796,901

(22) Filed: Mar. 9, 2004

(51) Int. Cl. A61B 17/00

(52) U.S. Cl. 606/190

(57) ABSTRACT

A surgical dissector comprising an elongate shaft having a proximal end and a distal end. A blunt dissection tip is positioned on the distal end of the elongate shaft. A light source emits a diffuse visible energy, such as white light, from the blunt tip. The shaft may take a variety of shapes, including being rigid, flexible, malleable, straight, bent, curved, articulated, and/or segmented. In addition, the shaft may include one or more functional components. The dissector can be used to locate the dissector tip by observing the visible energy passing through tissue. In addition, the visible energy passing through tissue may be used to differentiate tissue.

### BACKGROUND

The present invention relates to surgical tools, and more specifically to surgical dissectors. In the broadest sense, dissectors are used to cut apart or separate tissue. For instance, during an operation dissectors can be used to separate different structures along natural lines by dividing the connective tissue framework. Dissectors can take a wide variety of shapes and sizes. For example, some dissecting surfaces are blunt (e.g., rounded, fanned, or the like) while other dissectors have sharpened surfaces (e.g., needles, lances, blades, and the like). No one, however, has previously made or used dissector in accordance with the present invention.

### BRIEF SUMMARY

One example of the invention a surgical dissector comprising an elongate shaft having a proximal end and a distal end. A blunt dissection tip is positioned on the distal end of the elongate shaft. A light source emits a visible energy, such as a diffuse and/or unfocused white light, from the blunt tip. The shaft may take a variety of shapes, including being rigid, flexible, malleable, straight, bent, curved, articulated, and/or segmented. In addition, the shaft may include one or more functional components.

Another example of the invention is a method of separating a first tissue from a second tissue. A blunt tipped dissector is positioned near the first and second tissues. The first and second tissues are separated by moving the blunt tipped dissector between the first and second tissues, wherein the first or second tissues obstruct the operator's sight of the dissector tip. A diffuse light is emitted from the dissector tip while positioned between the first and second tissues. The tip of the dissector is visually located by observing the light passing through the obstructing tissue.

Yet another example of the invention is a method of separating a first tissue from a second tissue. A blunt tipped dissector is positioned near the first and second tissues. The first and second tissues are separated by moving the blunt tipped dissector between the first and second tissues. A diffuse light is emitted from the blunt tip of the dissector while positioned between the first and second tissues. Tissue is differentiated by observing the light passing through the first or second tissues.

The foregoing brief description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### BRIEF DESCRIPTION OF DRAWINGS

While the specification concludes with claims which particularly point out and distinctly claim the invention, it is believed the present invention will be better understood from the following description taken in conjunction with the accompanying drawings, in which like reference numerals identify the same elements and in which:

FIG. 1 illustrates an example of a dissector;

FIG. 2 illustrates another example of a dissector; and

FIG. 3 illustrates a partial cross-sectional view of a portion of the dissector shown in FIG. 2.

### DETAILED DESCRIPTION

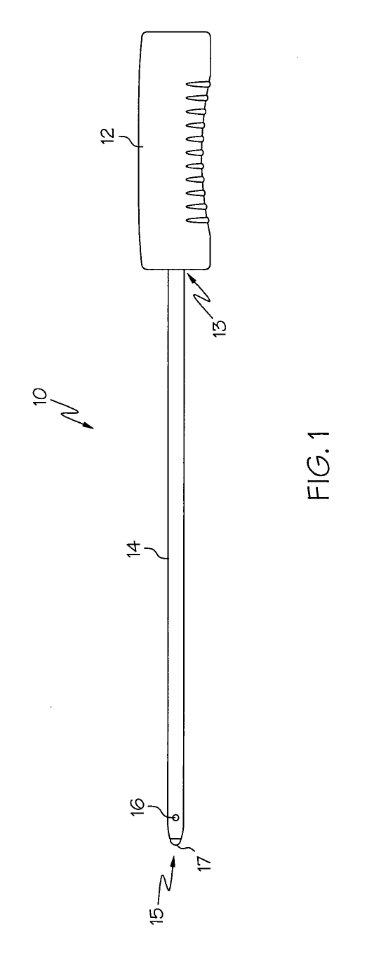

FIG. 1 illustrates an example of a dissector 10. The dissector 10 includes and elongate shaft 14 having a proximal end 13 and a distal end 15. A handle 12 is connected to the shaft 14 at the proximal end 13. In the present example, the shaft 14 is made from stainless steel, but numerous other materials known in the art may also be employed. The shaft 14 and has a circular cross section along its length and the distal end 15 is a blunt and rounded tip, which tip may be smooth or rough. Any portion of the shaft 14 can be used for dissecting tissue. It should be appreciated, however, that variable cross-sectional shapes are also contemplated, such as a fanned or flatted portions. In addition, the distal end 14 could have numerous other geometries, such as a Y-shaped tip.

As shown in this example, the shaft is substantially straight; however, the shaft 14 can take a variety of alternative shapes. For instance, the shaft 14 could be bent, curved, arced, undulated, helical, twisted, and the like. Further, the shaft 14 could be moveable, such as having one or more articulated joints or multiple segments. In addition, the shaft 14 could be rigid, flexible or malleable, either along its entire length or only along a portion. The shaft 14 includes an optional hole 16 so that sutures or other devices may be attached. In an alternative embodiment, the distal end 15 includes a step or barb onto which an elastomeric tube could be connected. The shaft 14 may also include one or more functional components to facilitate dissection, such as a grasper, an inflatable balloon, an expanding cage or arm, retractors, an ultrasonic emitter, a retractable sharped surface, an endoscope, a port for water jet dissection, a guide wire, a oxygen content sensor, a working lumen, a fixed or rotating knurled ball, or other components known in the art. The functional components can be integral to the dissector 10 or could be separable, such as removable or interchangeable tips.

A light source 17 is positioned at the distal end 15 of the shaft 14. The light source 17 emits a visible energy. In the present example the visible energy is a diffuse and substantially unfocused. The wavelength of the visible energy may vary, including for instance being substantially white, green, red, or other color. The light source 17 in this example takes the form of an light emitting diode (LED) positioned on the distal tip of the shaft 14. Alternative lights sources may also be used, including without limitation incandescent, fluorescent, laser, infrared and the like. The visible energy can originate directly from the light source 17 or can originate from a position remote to the distal end 15 (e.g., in the shaft 14, handle 12, or external to the dissector). For instance, the light can be delivered to the distal end 15 via fiber optics or a light pipe. While the light source 17 in the present example emits light from a point positioned on or near the distal end 15, it is also contemplated that light could be emitted from multiple points or from an area, such as along a segment of the shaft 14.

The visible energy has sufficient luminous intensity to pass through tissue. Suitable luminous intensity will vary depending upon the tissue being dissected. Some exemplary ranges of luminous intensity include between about 20 lux and about 50,000 lux, 300 lux and about 1500 lux, between about 500 lux and about 1500 lux, and between about 700 lux and about 1300 lux. Note that these ranges are merely illustrative and not limiting. The light source 17 here is powered by a battery positioned in the handle 12, but it could be powered using different configurations such as a remote tethered power source.

One illustrative use of the dissector 10 is to separate two adjacent tissues. The distal end 15 is position at the junction of the two tissues. As the shaft is moved between the tissues, the two tissue separate and become dissected. By laterally moving the shaft, a wider dissection can be achieved. In many cases, one or both of the tissues being dissected may obstruct the surgeon's line of sight, such that they cannot visually identify the location of the distal end 15. In such situations, the locating the distal end 15 can be located by observing the diffuse visible energy passing through the obstructing tissue. Accordingly, the operator will have better control and accuracy while dissecting. In addition, by observing the visible energy passing through the tissue the surgeon can differentiate between different tissues. The light source 17 can continuously emit, periodically emit (e.g., a slow or rapid sequence such as with a strobe), or selectively emit the visible energy (e.g., activate the light source only when desired). Being able to locate the distal end 15 which would otherwise be visually obstructed and/or being able to differentiate tissue is particularly useful when dissecting fragile tissue or near sensitive organs.

In addition to transillumination of tissue, the visible energy can be used to directly illuminate a surgical area. For instance, a surgeon may desire to illuminate a surgical field. In one variation, the shaft 14 has a lumen and the distal end 15 is transparent. In such embodiment, an endoscope can be threaded through the lumen and the surgeon may visualize a patient's anatomy from the perspective of the distal end 15 while being illuminated by the light source 17.

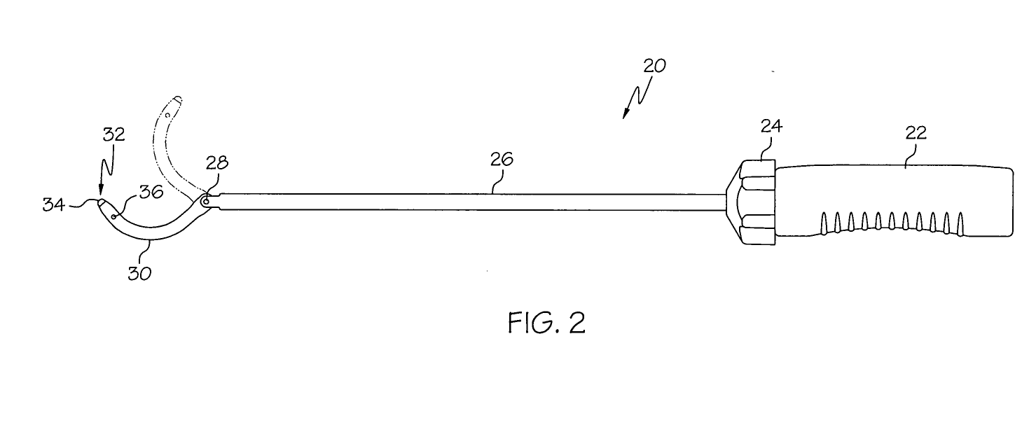

FIG. 2 illustrates another example of a dissector 20. The dissector 20 comprises an elongate shaft 26 with a handle 22 connected to the proximal end of the shaft 26. The shaft 26 is articulated and includes an arcuate and elongate segment 30 distal the joint 28 and a substantially straight segment proximal the joint 28. The segment 30 has blunt and rounded distal end 32, and includes an optional suture hole 36. The segment 30 pivots about a joint 28. In the present example, the segment 30 pivots about a single axis of rotation, but more complicated joints may also be employed. A knob 24 is positioned on handle 22 that actuates and controls the position of the segment 30 by manually rotating the knob 24. The present figure illustrates two exemplary angular positions. The segment 30 shown in solid is positioned in a “straight” or “back” position where the distal end 32 is substantially aligned with aligned with axis of the shaft 26 (i.e, at 0°). As shown in phantom, the segment 30 is in a “bent” or “forward” position where the distal end 32 is positioned at about 75° from the axis of the shaft 26. The segment 30 can pivot to any position between the extremes of 0°-75°. Alternatively, the segment 30 can be pivoted outside that range (i.e., less than 0° and/or greater than 75°). For instance, one embodiment pivots between −30° and 140°.

A light source 34 emits visible energy from the distal end 32 of the segment 30. The light source 34 in this example emits a substantially unfocused and diffuse light. While a variety of different light sources 34 may be employed, the present embodiment uses a model NSPW500BS white LED produced by NICHIA positioned on the distal end 32. A battery in the handle 22 powers the light source 34.

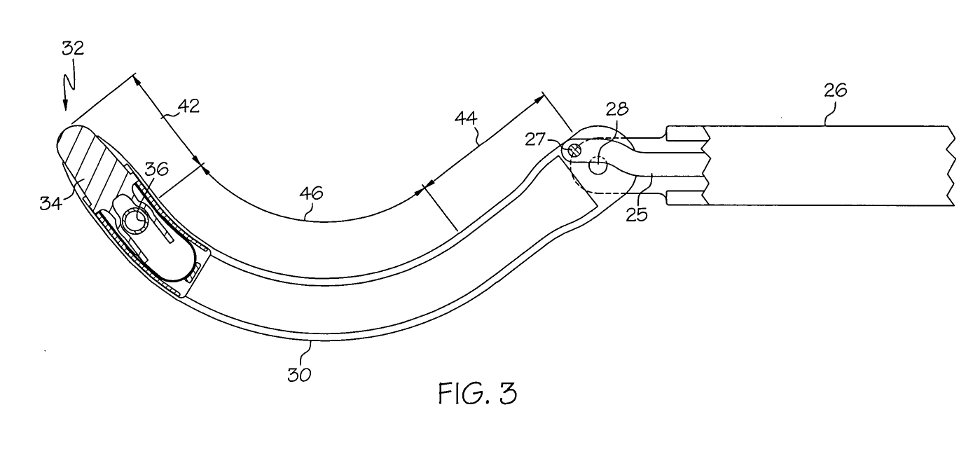

FIG. 3 illustrates a partial cross-sectional view of the dissector 20. The light source 34 is partially encased within the segment 30 wall and is exposed to define the blunt tip geometry of the distal end 32. A connection rod 25 is positioned in the shaft 26 and connects to the proximal end of the segment 30 with a pin 27 offset from the axis of rotation of the joint 28. The other end of the rod (not shown) is connected to a worm screw that engages a threaded nut connected to the knob 24. Accordingly, the operator can manually rotate the knob 24 which axially moves the rod 25, which in turn pivots the segment 30. One advantage of this embodiment is that the after the surgeon releases the knob 24, the angular position of the segment 30 relative the shaft 26 remains secure and relatively rigid. While the present actuation arrangement has certain advantages, other actuation arrangements known in the art may also be used, including without limitation scissors-type handles, rolling wheels, slide levers, spring mechanisms.

While the geometry of the arcuate segment 30 may vary significantly based on the targeted anatomy, the following describes the geometry of present example. The segment 30 in the present example has a smooth outer surface and a substantially circular cross-sectional shape that tapers slightly toward the distal end 32. The nominal diameter is about 3/16 inch, but a variety of other diameters may be used, including without limitation diameters ranging from 0.5 to 0.075 inches. The length of the segment 30 measured from the distal end 32 to the joint 28 ranges from about 2 to 2.5 inches, but the length may be extended outside this range depending upon the intended medical procedure. For instance, the length may also be between about 0.5 to 4 inches. The arcuate shape of the segment 30 in this example includes an arc portion 46, a proximal linear portion 44, and a distal linear portion 42. The radius of the arc portion 46 shown here is about 1 inch and swept about 90°; however, other arc geometries may be used, including without limitation arc radii ranging from 0.25 to 3 inches and swept 300 to 180°. The proximal linear portion 44 here is about 0.5 inches long and the distal linear portion is about 0.25 inches long. The dimensional range of the linear portions 42, 44 may also be varied substantially. Naturally, the foregoing geometries are merely illustrative and should not be considered limiting.

The dissector 20 of the present example is well-suited for separating and/or isolating a variety of tissues, during both open and/or minimally invasive procedures. Some exemplary procedures include, without limitation:
Isolate pulmonary arteries and branches;
Isolate pulmonary veins and branches;
During billiary surgery with gall bladder, separating the vein from artery and/or separating the bile duct from the vascular pedicle;
Isolate aorta, such as for retroperiteneal isolation of thoracic or abdominal aorta;
Isolate renal pedicle;
Isolate illiac vessel;
Isolate femoral artery from vein;
Isolate arch vessels;
Isolate carotids;
Isolate rectum from pelvic floor through peritoneum; and
Isolate other tubular structures from connective tissue.

The following describes an exemplary procedure using the dissector 20 to separate the left or right pair of pulmonary veins adjacent the left atrium. The procedure may be performed during open or minimally invasive surgery. With the segment 30 in a substantially straight position, the distal end 32 of the segment 30 is positioned adjacent the junction of one of the pulmonary veins (superior or inferior) and the left atrium. The distal end 32 is advanced around the posterior of the pair of pulmonary veins while simultaneously changing the angular position of the segment 30 in the forward direction. The distal end 32 continues to advance until it emerges beyond the other adjacent pulmonary vein (the inferior or superior, as the case may be). The advancement of the distal end separates the pair of pulmonary veins from the pericardial reflections, thus creating a path between the pulmonary veins and the pericardium. The path can be widened by gently rotating back and forth the handle 22 while the segment 30 is in an articulated position, which will sweep the segment 30 and further separating the tissue and widen the path.

If the light source 34 is used, it has several useful benefits during the procedure. One benefit is to illuminate the surgical area during the initial approach and positioning of the distal end 32. Another benefit is to locate the distal end 32 during the procedure. While advancing, the distal end 32 is often obstructed from sight by the surrounding tissue. The light emitting from the light source 34 passes through the obstructing tissue and the surgeon can visually locate the distal end 32 by observing such light. Still another benefit of the light source 34 is to differentiate between the various tissue. By observing light passing through tissue, the surgeon can discern if the distal end is approaching or contacting targeted or untargeted tissue. Accordingly, the surgeon has greater control and accuracy while dissecting the area.

One reason to dissect the pulmonary veins is as part of a procedure to treat atrial fibrillation. After the distal end 32 emerges beyond both pulmonary veins, further advancement and articulation will expose the distal end 32. A guide is then attached to the segment 30. For example, the guide may take the form of a suture or umbilical tape threaded through the hole 36. In another example, the guide may be a flexible catheter (such as a BARDIA urethral catheter) fitted over the distal end 32. The segment 30 is then reversed back through the path while pivoting the segment 30 in the backward direction, thus threading the guide through the path resulting in a sling around the pulmonary veins. The guide is then attached to one jaw of a clamping ablation device (including without limitation the devices disclosed in U.S. Pat. No. 6,517,536). By pulling the other end of the guide, the jaw can be accurately positioned in the path and the pulmonary veins are interposed between the ablation jaws. The jaws can then be closed and the targeted tissue ablated.

Having shown and described various embodiments of the present invention, further adaptations of the methods and systems described herein can be accomplished by appropriate modifications by one of ordinary skill in the art without departing from the scope of the present invention. Several of such potential modifications have been mentioned, and others will be apparent to those skilled in the art. Accordingly, the scope of the present invention should be considered in terms of the following claims and is understood not to be limited to the details of structure and operation shown and described in the specification and drawings.

1. A surgical dissector, comprising:
a) an elongate shaft;
b) a handle connected to shaft;
c) an articulation joint on the shaft;
d) an arcuate and elongate segment distal the articulation joint and having a blunt distal end;
e) an actuator operable to pivot the segment; and
f) a light source emitting visible energy from the distal end of the segment.

2. The surgical dissector of claim 1, further comprising a hole positioned near the distal end of the arcuate segment.

3. The surgical dissector of claim 1, wherein the arcuate segment comprises an arc portion.

4. The surgical dissector of claim 3, wherein the arcuate segment further comprises a linear portion distal the arc portion.

5. The surgical dissector of claim 3, wherein the arcuate segment further comprises a linear portion proximal the arc portion.

6. The surgical dissector of claim 1, wherein the arcuate segment is between about 2 inches and 2.5 inches in length.

7. The surgical dissector of claim 1, wherein the shaft comprises a straight portion proximal the articulation joint.

8. The surgical dissector of claim 7, wherein the arcuate segment can pivot between a first position where the distal end of the segment is substantially aligned with axis of the straight portion, and a second position where the distal end of the segment is at an angle relative the axis of the straight portion.

9. The surgical dissector of claim 1, further comprising an actuation rod connected at one end to the segment and connected at the other end to the actuator.

10. The surgical dissector of claim 9, wherein the actuator comprises a knob on the handle.

11. The surgical dissector of claim 9, wherein the actuation rod is positioned in the shaft.

12. The surgical dissector of claim 1, wherein the visible energy is a diffuse and unfocused light.

13. The surgical dissector of claim 12, wherein the luminous intensity of the LED is greater than about 300 lux and less than about 1500 lux.

14. The surgical dissector of claim 1, wherein the light source is an LED.

15. A method for separating tubular structures from connective tissue with the surgical dissector of claim 1, comprising the steps of:
a) positioning the blunt distal end of the segment adjacent a tubular structure;
b) advancing the blunt end around the tubular structures to separate the tubular structures from the connective tissue; and
c) simultaneously pivoting the arcuate segment.

16. The method of claim 15, wherein the tubular structure is a blood vessel.

17. The method of claim 16, wherein the blood vessel is a pulmonary vein and the connective tissue is the pericardium.

18. The method of claim 17, wherein steps are part of a procedure for treating atrial fibrillation.

19. The method of claim 15, further comprising the step of visually locating the distal end of the arcuate segment by observing the visible energy passing through tissue.

20. The method of claim 15, further comprising the step of differentiating tissue by observing the visible energy passing through tissue.

21. A surgical dissector, comprising:
a) an articulated elongate shaft, the shaft comprising a joint, a rigid straight segment proximal the joint, and a rigid arcuate segment distal the joint;
b) an actuator operable to control the angular position of the arcuate segment relative the straight segment;
c) a blunt tip on the distal end of the arcuate segment; and
d) a light source emitting a diffuse light from the blunt tip.

22. The surgical dissector of claim 21, wherein the arcuate segment comprises an arc portion and a linear portion proximal the arc portion.

23. A surgical dissector, comprising an articulated elongate shaft having a smooth arcuate segment with a plurality of angular positions, said arcuate segment comprising an arc portion, a linear portion proximal the arc portion connected to a joint, and a blunt tip at the distal end of the arcuate segment emitting a diffuse light.

24. A surgical dissector of claim 23, further comprising a functional component means.
